(19) United States
(12) Patent Application Publication
(10) Pub. No.: US 2025/0027140 A1
(43) Pub. Date: Jan. 23, 2025

(54) METHOD FOR DETECTING NEURODEGENERATIVE DISEASE USING SHORT-CHAIN RNA

(71) Applicant: TORAY INDUSTRIES, INC., Tokyo (JP)

(72) Inventors: Chiori KASHIMURA, Kamakura-shi (JP); Hiroko SUDO, Kamakura-shi (JP)

(73) Assignee: TORAY INDUSTRIES, INC., Tokyo (JP)

(21) Appl. No.: 18/711,315

(22) PCT Filed: Nov. 18, 2022
(86) PCT No.: PCT/JP2022/042927

§ 371 Date: May 17, 2024

(30) Foreign Application Priority Data
Nov. 19, 2021 (JP) 2021-188468

(51) Int. Cl. C12Q 1/6837; C12Q 1/6851; G01N 33/563

(52) U.S. Cl. CPC C12Q 1/6837; C12Q 1/6851; G01N 33/563; G01N 2800/2821

(57) ABSTRACT

Disclosed is a method that enables detection of whether or not a subject is affected by a neurodegenerative disease, which method is simpler and more effective than conventional methods. This method includes the steps of: (a) preparing an extracellular vesicle fraction from a body fluid sample of the subject; (b) counting the number of extracellular vesicles contained in the extracellular vesicle fraction obtained in Step (a), to obtain the number of the extracellular vesicles; (c) measuring the total amount of short-chain RNA contained in all extracellular vesicles counted in Step (b), to obtain the total amount of short-chain RNA per extracellular vesicle; and (d) judging the subject as being affected by the neurodegenerative disease in a case where the total amount of short-chain RNA per extracellular vesicle obtained in Step (c) is larger than a total amount of short-chain RNA per extracellular vesicle obtained from a body fluid sample of a healthy individual.

### TECHNICAL FIELD

The present invention is a method of detecting whether or not a subject is affected by a neurodegenerative disease, using the total amount of short-chain RNA contained in extracellular vesicles in a body fluid sample of the subject.

### BACKGROUND ART

It is known that body fluids of organisms contain extracellular vesicles released from cells of organs and tissues, and that the extracellular vesicles circulate in the body.

Extracellular vesicles are vesicles secreted from various cells, having a diameter of about 10 nm to about 3000 nm. Since extracellular vesicles contain proteins, mRNAs, and short-chain RNAs such as microRNAs derived from the cells from which the extracellular vesicles were released, they have been reported to be useful as biomarkers. The extracellular vesicles which have a diameter of about 30 to 150 nm and which are derived from endosomes are exosomes. Exosomes are composed of a ceramide-rich lipid membrane, and produced in multivesicular endosomes. Exosomes are extracellularly secreted by fusion of the multivesicular endosomes with the cell membrane. Therefore, exosomes have membrane proteins that are endosome-specific markers, such as CD9 and CD63, on the membrane (Non-Patent Document 1).

Neurodegenerative diseases are diseases that cause various neuropathies due to weakening, functional decline, or death of nerve cells, and known examples of such diseases include Alzheimer's disease, dementia with Lewy bodies, Parkinson's disease, and amyotrophic lateral sclerosis (ALS). Detection of these diseases at an early stage may lead to early treatment, and may enable improvement of QOL of the patients.

The detection of the neurodegenerative diseases involves a variety of imaging tests such as MRI, FDG-PET, and DAT scan, each of which is highly invasive, requires a high testing cost, and is available only at limited measurement facilities. In view of this, biochemical diagnoses such as blood tests are desirable since they are less invasive and can be simply carried out.

Examples of blood biomarkers for neurodegenerative diseases include amyloid-β (Non-Patent Document 2), phosphorylated tau (Non-Patent Document 3), and microRNAs (Patent Document 1) for Alzheimer's disease. However, since each biomarker requires complex measurement and analysis algorithms using an advanced apparatus, such biomarkers have not been commonly used due to the lack of simplicity. Development of a diagnostic marker capable of simply detecting a neurodegenerative disease using a blood sample has been demanded.

### PRIOR ART DOCUMENTS

### Patent Document

[Patent Document 1] WO 2019/159884

### Non-Patent Documents

[Non-Patent Document 1] Lin J. et al. The Scientific World Journal, Volume 2015, Article ID 657086

[Non-Patent Document 2] Nakamura A. et al., Nature. 2018 Jan. 31, vol. 554, p. 249-254

[Non-Patent Document 3] Tatebe II.et al. Mol. Neurodegener., 2017 Sep. 4; 12(1):63

### SUMMARY OF THE INVENTION

### Problems to be Solved by the Invention

The present invention provides a method for simpler and more effective detection of the presence or absence of a neurodegenerative disease, which method eliminates imaging tests, which are burdensome to patients, and complex analyses using advanced measurement apparatuses.

### Means for Solving the Problems

In order to solve the above problem, the present inventors intensively studied to discover that the presence or absence of a neurodegenerative disease can be detected by measuring the total amount, per extracellular vesicle, of short-chain RNA contained in extracellular vesicles in a body fluid sample of a subject, and comparing the measured amount with that of a healthy individual, thereby completing the present invention based on the discovery.

Specifically, the present invention provides the following.

(1) A method of detecting whether or not a subject is affected by a neurodegenerative disease, the method comprising the steps of:
(a) preparing an extracellular vesicle fraction from a body fluid sample of the subject:
(b) counting the number of extracellular vesicles contained in the extracellular vesicle fraction obtained in Step (a), to obtain the number of the extracellular vesicles:
(c) measuring the total amount of short-chain RNA contained in all extracellular vesicles counted in Step (b), to obtain the total amount of short-chain RNA per extracellular vesicle: and
(d) judging the subject as being affected by the neurodegenerative disease in a case where the total amount of short-chain RNA per extracellular vesicle obtained in Step (c) is larger than a total amount of short-chain RNA per extracellular vesicle obtained from a body fluid sample of a healthy individual.

(2) The method according to (1), wherein in the Step (d), an average of total amounts of short-chain RNA per extracellular vesicle preliminarily obtained from body fluid samples of a plurality of healthy individuals is calculated, and the subject is judged as being affected by the neurodegenerative disease in a case where the total amount of short-chain RNA per extracellular vesicle obtained from the body fluid sample of the subject is not less than 1.5 times, and less than 100 times the average.

(3) The method according to (1) or (2), wherein in the Step (a), the prepared extracellular vesicle fraction contains an extracellular vesicle having a diameter of 30 nm to 200 nm.

(4) The method according to any one of (1) to (3), wherein in the Step (a), CD9, CD63, CD81, Tim4, or 1.1CAM protein is present on the surface of a prepared extracellular vesicle(s).

(5) The method according to any one of (1) to (4), wherein in the Step (b), the short-chain RNA has a length of 15 bases or more and 200 bases or less.

(6) The method according to any one of (1) to (5), wherein the short-chain RNA is a microRNA.

(7) The method according to any one of (1) to (6), wherein the body fluid sample is blood, serum, plasma, or cerebrospinal fluid.

(8) The method according to any one of (1) to (7), wherein in the Step (a), the extracellular vesicle fraction is prepared by a method selected from the group consisting of a centrifugation method, an immunoprecipitation method, a polymer precipitation method, a lipid affinity method, liquid chromatography, size exclusion chromatography, an ultrafiltration method, and combinations thereof.

(9) The method according to any one of (1) to (8), wherein in the Step (b), the number of the extracellular vesicles is counted by a tracking method, an antigen-antibody reaction method, or a flow cytometry method.

(10) The method according to any one of (1) to (9), wherein in the Step (c), the total amount of short-chain RNA contained in the extracellular vesicles is measured using a spectrophotometer, electrophoresis, microarray, PCR, or a DNA sequencer.

(11) The method according to any one of (1) to (10), wherein the neurodegenerative disease is Alzheimer's disease, dementia with Lewy Bodies, frontotemporal dementia, amyotrophic lateral sclerosis (ALS), or Parkinson's disease.

(12) A kit for detecting whether or not a subject is affected by a neurodegenerative disease by the method according to (1), the kit comprising:
means for preparing an extracellular vesicle fraction from a body fluid sample of the subject;
means for counting the number of extracellular vesicles contained in the prepared extracellular vesicle fraction, to obtain the number of the extracellular vesicles; and
means for measuring the total amount of short-chain RNA contained in all counted extracellular vesicles, to obtain the total amount of short-chain RNA per extracellular vesicle.

(13) The kit according to (12), wherein
the means for preparing the extracellular vesicle fraction from the body fluid sample of the subject comprises an immobilized antigen or antigen-binding fragment thereof, prepared by immobilization of an antibody that specifically binds to a target surface antigen on extracellular vesicles or immobilization of an antigen-binding fragment of the antibody:
the means for counting the number of extracellular vesicles contained in the prepared extracellular vesicle fraction, to obtain the number of the extracellular vesicles, comprises a labeled antibody or antigen-binding fragment of the antibody, which antibody specifically binds to a target surface antigen on extracellular vesicles: and
the means for measuring the total amount of short-chain RNA contained in all counted extracellular vesicles, to obtain the total amount of short-chain RNA per extracellular vesicle, comprises a microarray containing nucleic acid probes that hybridize with a plurality of known microRNAs.

(14) A system for detecting whether or not a subject is affected by a neurodegenerative disease by the method according to claim 1, the system comprising:
means for preparing an extracellular vesicle fraction from a body fluid sample of the subject;
means for counting the number of extracellular vesicles contained in the prepared extracellular vesicle fraction, to obtain the number of the extracellular vesicles; and
means for measuring the total amount of short-chain RNA contained in all counted extracellular vesicles, to obtain the total amount of short-chain RNA per extracellular vesicle.

The present invention described above also encompasses the following preferred embodiments.

(15) A method of detecting whether or not a subject is affected by a neurodegenerative disease, the method comprising the steps of:
(A) measuring the number of extracellular vesicles contained in a body fluid sample of a subject; and
(B) measuring the amount of short-chain RNA, preferably microRNA, contained in the extracellular vesicles whose number is counted in Step (A);
wherein the subject is likely to have been affected by the neurodegenerative disease in a case where the amount of short-chain RNA per extracellular vesicle is larger than that in a healthy individual.

(16) The method according to (15), wherein the extracellular vesicles are extracellular vesicles expressing at least one neuronal surface marker selected from the group consisting of 1.1CAM, NCAM, Enolase2, total Tau protein (MAPT). Glutamate receptor 1 (GRIA1), and Proteolipid protein 1(PLP1), preferably 1.1CAM protein.

(17) The method according to (15) or (16), wherein the subject is judged as likely to have been affected by the neurodegenerative disease in a case where the amount of short-chain RNA per extracellular vesicle obtained from the body fluid of the subject is not less than 1.5 times, and less than 100 times a preliminarily calculated average of the amount of short-chain RNA per extracellular vesicle obtained from body fluid samples of a plurality of healthy individuals.

(18) The method according to any one of (15) to (17), wherein the extracellular vesicles are also expressing at least one protein selected from the group consisting of CD63, CD9, CD81, and Tim4 on the surface.

(19) The method according to claim (18), wherein the Step (A) is carried out by collecting extracellular vesicles using an immobilized antibody or antigen-binding fragment of the antibody, prepared by immobilization of an antibody that specifically reacts with the neuronal surface marker expressed on the surface of the extracellular vesicles or immobilization of an antigen-binding fragment of the antibody, subsequently reacting the extracellular vesicles with a labeled antibody or antigen-binding fragment of the antibody: which antibody specifically reacts with CD63, CD9, CD81, or Tim4, and measuring a label bound to the extracellular vesicles.

(20) The method according to any one of (15) to (19), wherein the Step (B) is carried out by reacting a microarray containing nucleic acid probes that hybridize with a plurality of known short-chain RNAs, with short-chain RNAs in exosomes, and measuring the short-chain RNAs bound to the microarray.

(21) An apparatus included in the system according to (14), for preparing the extracellular vesicle fraction from the body fluid sample of the subject.

(22) An apparatus included in the system according to (14), for counting the extracellular vesicles contained in the prepared extracellular vesicle fraction, to obtain the number of the extracellular vesicles. (23) An apparatus included in the system according to (14), for measuring the total amount of short-chain RNA contained in all counted extracellular vesicles, to obtain the total amount of short-chain RNA per extracellular vesicle. (24) A program or a recording medium in which the program is written included in the system according to (14), for measuring the total amount of short-chain RNA contained in all counted extracellular vesicles. to obtain the total amount of short-chain RNA per extracellular vesicle.

### Effect of the Invention

The present invention enables less-invasive simple judgment of whether or not a subject is affected by neurodegeneration.

### BRIEF DESCRIPTION OF THE DRAWINGS

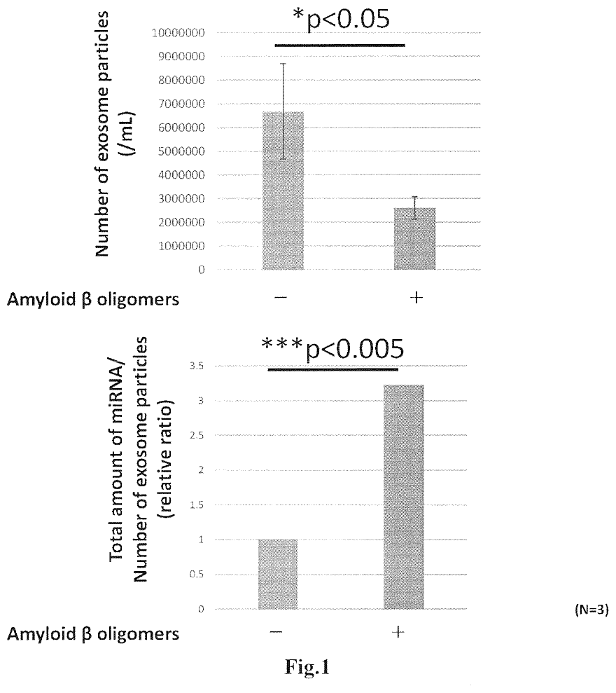

The upper panel in FIG. 1 shows a diagram illustrating the result of counting of the number of exosomies in the extracellular vesicle fraction (including exosomes and the like) prepared in Example 1. The lower panel in FIG. 1 shows a diagram illustrating the result of calculation of the relative ratio of the total amount of microRNA per exosome in the exosome fraction prepared in Example 1 as compared to a control.

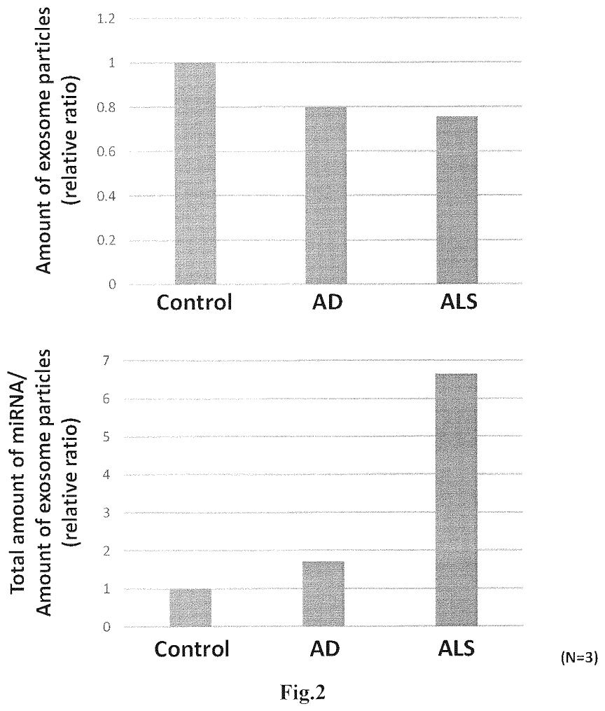

The upper panel in FIG. 2 shows a diagram illustrating the result of counting of the number of exosomes in the extracellular vesicle fraction (including exosomes and the like) prepared in Example 2, and determining its relative ratio to a control. The lower panel in FIG. 2 shows a diagram illustrating the result of calculation of the relative ratio of the total amount of microRNA per exosome in the exosome fraction prepared in Example 2 as compared to a control.

### MODE FOR CARRYING OUT THE INVENTION

The present invention is a method of testing whether or not a subject is affected by a neurodegenerative disease, the method comprising the following Steps (a) to (d).

Step (a): a step of preparing an extracellular vesicle fraction from a body fluid sample of the subject;

Step (b): a step of counting the number of extracellular vesicles contained in the extracellular vesicle fraction obtained in Step (a), to obtain the number of the extracellular vesicles;

Step (c): a step of measuring the total amount of short-chain RNA contained in all extracellular vesicles counted in Step (b), to obtain the total amount of short-chain RNA per extracellular vesicle; and

Step (d): a step of judging the subject as being affected by the neurodegenerative disease in a case where the total amount of short-chain RNA per extracellular vesicle obtained in Step (c) is larger than a total amount of short-chain RNA per extracellular vesicle obtained from a body fluid sample of a healthy individual.

The “subject”, to which the method of the present invention is to be applied, means a mammal, including primates such as humans, chimpanzees, and gorillas; pet animals such as dogs and cats: domestic animals such as cows, horses, sheep, and goats; rodents such as mice and rats: and animals kept in zoos. The subject is preferably a human. In particular, in cases where the subject is a mammal such as a human, and is affected by a neurodegenerative disease, the subject may be referred to as “patient”, and the patient is preferably a human. In the present invention, the “healthy individual” also means such a mammal including a human, and means an animal not affected by the neurodegenerative disease to be detected. The healthy individual is preferably a human.

In the present invention, neurodegeneration means a state where the structure and the function of neurons, which are nerve cells, are deteriorated. The most common examples of neurodegenerative diseases include Alzheimer's disease (Alzheimer's dementia), dementia with Lewy Bodies, frontotemporal dementia, corticobasal degeneration, Parkinson's disease, Huntington's disease, and amyotrophic lateral sclerosis (ALS). Alzheimer's disease may cause cognitive impairment; Parkinson's disease may cause a difficulty in a smooth motor function; and Huntington's disease may exhibit symptoms both in the cognitive function and the motor function.

Diagnosis of dementia, for example, Alzheimer's disease, dementia with Lewy Bodies, or frontotemporal dementia, is carried out by measuring a decrease in the volume of a particular part of the brain or a decrease in the cerebral blood flow, based on an imaging test such as computed tomography (CT), magnetic resonance imaging (MRI), positron emission tomography (PET), or single-photon emission computed tomography (SPECT). In some cases, biomarkers of dementia, such as amyloid β protein, tau protein, and phosphorylated tau protein contained in spinal fluid and blood are measured to aid the diagnosis. Dementia can also be diagnosed by a screening test by a neuropsychological test. One example of the screening test for dementia is a neuropsychological test represented by “Mini-Mental State Test” (MMSE). In an MMSE test, a score of 23 points or less out of 30 points is considered as an indicative of suspected dementia.

Diagnosis of Parkinson's disease includes not only the MRI test and the cerebral blood flow SPECT test described above, but also MIBG myocardial scintigraphy, which examines whether the function of the cardiac sympathetic nerve is deteriorated, and dopamine transporter scintigraphy (DAT scan (registered trademark)), which assesses the degree of degeneration and loss of the dopaminergic nerve. There is also a test that measures aggregates of α-synuclein protein in a body fluid such as spinal fluid.

Diagnosis of ALS uses tests for examination of the presence or absence of upper and lower motor neuronal impairments, such as the head MRI test and the spinal MRI test, which are useful for exclusion diagnosis, and also needle electromyography for examination of neurogenic changes and denervation findings, the peripheral nerve conduction velocity test for exclusion diagnosis, the motor-evoked potential test by central nerve magnetic stimulation, and evaluation tests for the respiratory muscle forced vital capacity, overnight oxygen saturation measurement, and arterial blood gas analysis). For ALS screening, neuropsychological tests such as the Edinburgh Cognitive and Behavioral ALS Screen (CAS) and the ALS Cognitive Behavioral Screen (ALS-CBS) are used in some cases. In the ECAS test, a score of 105 points or less out of a total score value of 136 points suggests possible functional impairment, and is considered as an indicative of suspected ALS. In the ALS-CBS test, a score of 36 points or less suggests behavioral impairment, and a score of 32 points or less suggests frontotemporal dementia.

In Huntington's disease, atrophy of the part called caudate nucleus in the basal ganglion is found by a head CT test and MRI test, and its progression leads to whole-brain atrophy, and enlargement of the anterior horn of the lateral ventricle as a result, which enables the diagnosis. In addition, a frontotemporal-lobar-type decrease in the blood flow is often found in the cerebral blood flow SPECT test.

Since Huntington's disease occurs due to genetic abnormality, genetic diagnosis by the PCR method or the like is used for its definitive diagnosis. In cases where the number of CAG repeats in the IITT gene is not more than 26, the patient is diagnosed as normal. In cases where the number is not less than 36, the patient is diagnosed as having a risk of development of Huntington's disease. In cases where the number is not less than 40, the patient is diagnosed as having a very high risk of development of Huntington's disease.

In the present invention, the term “affected by a neurodegenerative disease” means that a subject is suffering from any of the neurodegenerative diseases described above. The term “not affected by a neurodegenerative disease” means that a subject is not suffering from any of these diseases. The term “healthy individual” means a subject who is not suffering from any neurodegenerative disease. More specifically, in the present invention, a healthy individual means, for example, a subject for whom no abnormality is found in the above-described imaging tests such as CT, MRI, PET, SPECT, MIBG myocardial scintigraphy, and dopamine transporter scintigraphy, a subject whose protein biomarkers such as amyloid β, tau, phosphorylated tau, and synuclein in spinal fluid and blood show normal values, a subject whose number of CAG repeats in the HTT gene is not more than 26, a subject with a score of more than 23 points in the MMSE test, a subject with a score of more than 105 points in the ECAS test, or a subject with a score of more than 36 points in the ALS-CBS test.

The term “detection” in the present invention may be replaced by the term test, measurement, or detection: or assistance or aid for these. Further, the term “judgment” or “evaluation” as used in the present invention has a meaning including assisting or aiding of decision, diagnosis, or evaluation based on a test result or measurement result.

The term “P” or “P-value” used in the present description means a probability at which a statistical tex shows an extreme statistic as compared to a statistic calculated from actual Jata under the null hypothesis. Therefore, the smaller the “P” or “P-value”, the more significant the difference between the values to be compared.

The “body fluid sample” to be detected in the present invention means a liquid biological mater al in which the amount of the short-chain RNA to be measured by the preser t invention may change depending on the presence or absence of neurodegeneration, the progression of neurodegeneration, production of a therapeutic effect on neurodegeneration, or the like. Specific examples of the body fluid sample may include body fluids such as cerebrospinal fluid, bone marrow fluid, blood, urine, saliva, sweat, lymph, tissue exudate, and secretory fluid; and serum and plasma prepared from blood. The body fluid sample also means a biological sample extracted from these, such as a sample containing a transcription product including RNA or micioRNA.

The present invention is directly or indirectly used for detecting the presence or absence of a neurodegenerative disease in a subject: for diagnosing the development, occurrence, and degree of progression of a neurodegenerative disease, diagnosing whether or not a neurodegenerative disease is improved or the degree of the improvement, or diagnosing the sensitivity to a treatment for a neurodegenerative disease: or for screening of a candidate substance useful for prevention, improvement, or treatment of a neurodegenerative disease.

Each step of the method for detecting a neurodegenerative disease of the present invention is described below.

### 1. Step (a)

Step (a) in the present invention is a step of preparing an extracellular vesicle fraction from a body fluid sample of a subject.

Extracellular vesicles are secreted from cells, and have a lipid bilayer membrane encapsulating their contents. Extracellular vesicles are derived from the cells from which they are secreted, and, when they are released to the extracellular environment, they may contain biological substances such as genes including RNA and DNA, and proteins, in the vesicles. Extracellular vesicles are known to be contained in body fluids such as blood, serum, plasma, cerebrospinal fluid, and lymph.

Specific examples of extracellular vesicles include exosomes, microvesicles, and apoptotic vesicles. Exosomes are also called “exosomes” or “ectosomes”, and have a diameter of about 30 to 150 nm. Since exosomes are derived from endosomes, they have membrane proteins such as CD9 and CD63, which are endosome-specific markers, on the membrane. Microvesicles are also called “microparticles” or “microvesicles”, and have a diameter of about 100 to 1000 nm. After budding of microvesicles from the cell membrane, the constricted parts are cut off by, for example, the action of the ESCRT complex, to release the microvesicles. Apoptotic vesicles have a diameter of 1000 to 3000 nm, and are produced by rapid fragmentation of cells during apoptosis.

The extracellular vesicle fraction prepared in the Step (a) is a fraction that contains biological vesicles having a size within the range of 30 nm to 200 nm. The extracellular vesicle fraction may be a fraction that preferably contains extracellular vesicles having a diameter of 20 nm or more and 1000 nm or less, more preferably contains biological vesicles having a diameter of 10 nm or more to 3000 nm or less, which is known to be a size of common extracellular vesicles. The extracellular vesicle fraction used may be obtained by performing purification to remove a fraction containing larger cells (with a diameter of 6 to 25 μm) and/or smaller cell fractions or debris (with a diameter of not more than 10 nm).

Examples of the method of preparing or purifying the extracellular vesicle fraction include methods utilizing the size or density of the extracellular vesicles, or utilizing a special protein present on the surface of the extracellular vesicles. Specific examples of the method include centrifugation method, immunoprecipitation method, polymer precipitation method, lipid affinity method (affinity method), liquid chromatography (for example, high performance liquid chromatography), size exclusion chromatography, and ultrafiltration method. A plurality of these methods may be carried out in combination. These methods per se are known, and can be easily carried out using commercially available kits and apparatuses as described below.

The centrifugat on method is a method in which a centrifugal force is applied to a sample to separate or fractionate components constituting the sample. In particular, ultracentrifugation is the most classical gold standard. For example, in a method for obtaining an extracellular vesicle fraction by ultracentrifugation, a supernatant, serum, or plasma sample of cells is centrifuged at 10,000 g for 30minutes, and then the resulting supernatant is extracted. Thereafter, centrifugation is performed at 100,000 g for 70 minutes, and the resulting precipitate is washed by addition of phosphate buffer or the like, followed by performing centrifugation again at 100,000 g for 70 minutes. As a result, an extracellular vesicle fraction can be obtained as a precipitate (Clotilde Thery et al., Isolation and Characterization of Exosomes from Cell Culture Supernatants and Biological Fluids. Current Protocols in Cell Biology (2006) 3.22.1-3.22.29). Taking into account the purity and the yield of the extracellular vesicle fraction to be obtained, the centrifugation method can be carried out by application of a protocol in which the centrifugal force, the centrifugation time, the number of times of washing, and the like are optimized. Thus, an extracellular vesicle fraction can be suitably obtained even by a method other than the method described above.

The immunoprecipitation method is an immunochemical technique that uses an antibody that recognizes a particular antigen, to selectively separate and analyze the target antigen or a molecule that shows affinity to the antigen, from a mixture. In the present invention, a particular antigen or marker present on the surface of extracellular vesicles can be utilized. Common examples of such an antigen or a marker on extracellular vesicles that may be used include CD9, CD63, CD81, HSP70, ALIX, ANXAS, TSG101, FLOT-1, ICAM1, calnexin (CANX), CXCR4, EpCAM, Vimentin, and Tim4. The neuronal surface markers L1CAM, NCAM, enolase 2, total tau protein (MAPT), glutamate receptor 1 (GRIA1), and proteolipid protein 1 (PLP1) may also be used. Separation or purification of extracellular vesicles by immunoprecipitation is possible by binding them to magnetic beads to which an antibody of these markers is bound. The separation or purification can be carried out using, for example, the “Exo-Flow Exosome IP Kit”, manufactured by System Biosciences. L. C. or the ExoCap (trademark) Streptavidin Kit (MBL). Preferably, extracellular vesicles expressing both at least one of the above-described extracellular vesicle markers and at least one of the above-described neuronal surface markers are collected.

The polymer precipitation method is a method that enables precipitation of extracellular vesicles simply by a single centrifugation operation using a polymer based on a relative specific gravity. The method may be carried out using, for example. “ExoQuick”, manufactured by System Biosciences, LLC.

The lipid affinity method is also called the binding affinity method or affinity method, and utilizes reaction of a molecule (ligand) that irreversibly binds to a target subject, to separate or purify a target substance or a complex thereof. For example, a substance that binds calcium-dependently to phosphatidyl serine present on the lipid bilayer membrane surface of extracellular vesicles can be used to separate the extracellular vesicles, and then a chelating agent can be used to cancel the bond.

The method may be carried out using, for example, the “MagCaptur (trademark) lixosome Isolation Kit PS”, manufactured by Fujifilm Wako Pure Chemical Corporation.

Liquid chromatography is a method in which a solvent containing a sample dissolved therein is passed through a tube to separate components based on a difference in the migration velocity depending on the molecular size and the electric charge. In particular, high-performance liquid chromatography is a method using, as the mobile phase, a liquid pressurized at high pressure. The high-performance liquid chromatography can be carried out using, for example, “Shim-pack Scepter Chemistry”, manufactured by Shimadzu Corporation.

Size exclusion chromatography is commonly called gel filtration chromatography, and enables separation or purification of extracellular vesicles from impurities such as proteins using a complex matrix packed in a column. Size exclusion chromatography has an advantage that the extracellular vesicles can be obtained without affecting the shape and function of the vesicles. The size exclusion chromatography can be carried out using, for example, the “PURE-EV column”, manufactured by HansaBioMed Life Sciences Ltd., or “EVSecond L70”, manufactured by GL Sciences Inc.

The ultrafiltration method is a technique in which a pressure is applied to a solution on one side of a dialysis membrane to extrude very small molecules such as water and salts in the solution to the other side of the membrane, to separate or purify a remaining substance. In the present invention, the method is applicable to the separation of extracellular vesicles. Since this method has an excellent ability to eliminate biomolecules smaller than extracellular vesicles, it is suitable for separation or purification of extracellular vesicles contained, for example, in a solution preliminarily prepared by removing macromolecules from serum or plasma, or in a supernatant of cells prepared by serum-free culture. In this case, the method can be carried out. for example: using “Amicon (registered trademark) Ultra-15”, manufactured by Merck.

### 2. Step (b)

Step (b) in the present invention is a step of counting the number of extracellular vesicles contained in the extracellular vesicle fraction obtained in Step (a), to obtain the number of the extracellular vesicles.

The tracking method is a method in which a high-sensitivity camera is used to analyze the trajectories of individual particles due to Brownian motion, to calculate the migration velocities of the individual particles in a sample suspension, followed by analyzing the particle sizes based on the calculated velocities. Specifically, the method can be carried out using “NanoSight”, manufactured by Malvern Panalytical, Spectris.

The antigen-antibody reaction method is a method in which a relative amount of extracellular vesicles, is measured by sandwich ELISA using an antibody against a surface marker specific to extracellular vesicles. The method can be carried out using, for example, the “ExoTEST (trademark) Ready to use ELISA kit”, manufactured by Hans&BioMed Life Sciences Ltd. Further, after capturing extracellular vesicles by antibody-antigen reaction. “ExoCounter”, manufactured by JVC KENWOOD Corporation, or the like can be used to enable absolute quantification by digital measurement using nanobeads. Further, the activity of an enzyme contained in the extracellular vesicles, such as the acetyl-CoA acetyleholinesterase activity, can be used for rough quantification of the number of extracellular vesicles. Specifically, the quantification can be carried out using the “EXOCET Exosome Quantitation Kit”, manufactured by System Biosciences, LLC.

In the flow cytemetry method, extracellular vesicles are stained with a fluorescent dye, and then bound to a carrier such as beads utilizing antibody-antigen reaction or the like, followed by performing measurement using a fluorescence microscope. The method can be carried out using, for example, the “PS Capture (trademark) Exosome Flow Cytometry Kit”, manufactured by Fujifilm Wako Pure Chemical.

### 3. Step (c)

Step (c) in the present invention is a step of measuring the total amount of short-chain RNA contained in all extracellular vesicles counted in Step (b), to obtain the total amount of short-chain RNA per extracellular vesicle.

In the present description, “RNA” includes any of total RNA, mRNA, rRNA, microRNA, SiRNA, shRNA, piRNA, snoRNA, snRNA, and non-coding RNA.

In the present irvention, the short-chain RNA means RNA having a length of 15 bases to 200 bases. Examples of the short-chain RNA include microRNA, SiRNA, shRNA, piRNA, snoRNA, and snRNA. Among these, microRNA is preferred.

Among the short-chain RNAs, unless otherwise specified, the microRNA is a microRNA precursor, which is a transcription product having a hairpin structure, or an RNA produced by cleavage by a dsRNA-cleaving enzyme having the RNase III-cleaving activity after the transcription. The micro RNA is incorporated into a protein complex called RISC. MicroRNAs are involved in translational repression of mRNA. MicroRN As are typically non-coding RNAs of 15 to 30 bases.

In the present description, the microRNA includes “immature microRNA” and “mature microRNA”. The immature microRNA means a microRNA that has not yet become the later-mentioned mature microRNA. The immature microRNA includes “microRNA precursor”.

In the present description, “mature microRNA” is a microRNA that can be incorporated into RISC after cleavage by the dsRNA-cleaving enzyme described above.

A microRNA in the present invention is not limited to a microRNA itself having a particular bas: sequence, and also encompasses a precursor of the microRNA (pre-microRNA, pri-microRNA), and a microRNA having a biological function equivalent to that of the microRNA having the particular base sequence, for example, a microRNA that is a homolog thereof (that is, a homolog or an ortholog), a mutant due to genetic polymorphism or the like, or a derivative, of the microRNA having the particular base sequence. When a mature microRNA is produced by cleavage from an RNA precursor having a hairpin structure, the cleavage may result in truncation or extension of the ends of the sequence by one or several bases, or may cause base substitution, to produce a mutant that is called isomiR (Morin R D. et al., 2008, Genome Res., vol. 18, pp. 610-621). Such a specific microRNA that is a precursor, a homologous RNA, a mutant, a derivative, or an isomiR can be identified by, for example, “miRBase” (version 22), “miRBase” (version 22) is a web-based database that provides base sequences, annotations, and prediction of target genes of microRNAs. The microRNAs deposited in “miRBase” (http:/www.mirbase.org/) are limited to microRNAs that have already been cloned, or that have been shown to be expressed in the body and undergo processing. The microRNA in the present invention may also be a gene product of an miR gene, and such a gene product encompasses a mature microRNA (such as the above-described non-coding RNA of 15 to 30 bases involved in translational repression of mRNA) or a microRNA precursor (such as the above-described pre-microRNA or pri-microRNA).

In the present invention, the term “total amount of short-chain RNA” means the number of molecules or the copy number of the short-chain RNA. The amount of short-chain RNA can be measured, for example, by a spectrophotometer. electrophoresis, microarray, PCR, or a DNA sequencer. In this case, the amount is the total detectable amount of RNAs that are short chains irrespective of their individual RNA sequences. The “total amount of short-chain RNA” means the total amount of short-chain NA detected by the measurement method employed. Therefore, for example, in cases where “3D-Gene” (registered trademark), which is a microarray, is used as the measurement method as in the following Examples, a plurality of microRNAs may be simultaneously detected, and the sum of the measured values may be regarded as the total amount of short-chain RNA in the present invention.

Regarding the method of obtaining RNA from the extracellular vesicles, for example, the commonly used acidic phenol method (Acid (juanidinium-Phenol-Chloroform (AGPC) method) may be used, or an RNA extraction reagent containing acidic phenol, such as “Trizol” (registered trademark) (Life Technologies) or “Isogen” (Nippon Gene) may be used. Alternatively, a kit such as the “miRNeasy Mini Kit” (Qiagen) may be used, or an RNA extraction reagent in the “3D-Gene (registered trademark) RNA extraction reagent from liquid sample kit” (Toray Industries, Inc.) may be used. The method is, however, not limited to these.

The measurement of the amount of short-chain RNA may be carried out, for example, by a method in which the whole obtained RNA is used as a sample to specifically measure short-chain RNA by the use of electrophoresis, microarray, PCR, a DNA sequence: or the like. The measurement may also be carried out by a method in which short chain RNA is preliminarily collected from an RNA sample using an ultrafiltration column or the like, followed by measuring the short-chain RNA by a method using a spectrophotometer or the like.

The electrophoresis method is a method in which an electric field is applied to a gel matrix through which a measurement target (nucleic acid, protein or the like) passes, to separate the measurement target based on a difference in the mobility depending on the size, electric charge, and structure. This method is especially useful for analysis of the base sequence of nucleic acid. Measurement of short-chain RNA is possible by the use of polyacrylamide gel electrophoresis (SDS-PAGE) under denaturing conditions in the presence of a surfactant such as SDS, or by the use of capillary electrophoresis in cases of measurement with a smaller amount of sample. In particular, the latter enables measurement of the amount of short-chain RNA by using, for example, “2100 Bioanalyzer”, manufactured by Agilent Technologies, Inc.

A microarray is a relatively compact apparatus capable of measurement of a plurality of small target substances, especially nucleic acids (DNA, RNA), at the same time. It is also called nucleic acid array, nucleic acid chip, or DNA chip. In the present invention, the total amount of short-chain RNA can be measured by using an array specialized for microRNA and piRNA, which are short-chain RNA. For example, the amount of short-chain RNA can be calculated from the sum of the expression levels of microRNA and piRNA. By using “3D-Gene” (registered trademark), manufactured by Toray Industries, Inc., a microarray for microRNA manufactured by Agilent Technologies, Inc., or the like as such a microarray, the amount of short-chain RNA can be obtained. In the Examples below, the total amount of microRNA was measured using “3D-Gene” (registered trademark), manufactured by Toray Industries, Inc. “3D)-Gene” (registered trademark) is a microarray containing DNAs complementary to 2632 kinds of microRNAs, which cover the majority of known human microRNAs. Therefore, “3D-Gene” (registered trademark) can be preferably used in the present invention. In cases where a microarray is used, the microarray preferably contains as much DNAs complementary to known human microRNAs as possible. The microarray contains preferably not less than 1000, more preferably not less than 2000, still more preferably not less than 2600 kinds of DNAs complementary to known human microRNAs. The version of the microRNAs that are complementary to the DNAs contained in “3D)-Gene” (registered trademark) is described at https://www.3d-gene.com/products/dna, and details of the version are deposited at miRBase release 22 (http://www.mirbase.org/). The amount of the microRNAs bound to the microarray can be measured by fluorescently labeling the microRNAs in advance, hybridizing the microRNAs with the DNAs on the microarray, and then quantifying the fluorescent label immobilized on the microarray. This can be simply carried out using a kit and an apparatus that are commercially available, as specifically described in the Examples below.

Regarding the POR (Polymerase Chain Reaction), a method such as multiplex PCR in which a plurality of target short-chain RNAs are simultaneously detected is especially useful in the present invention. There are kits that enable simultaneous detection of a plurality of microRNAs. Examples of such kits that may be used include “miRCURY RNA miRNA PCR Assays” and “miScript miRNA POR Arrays”, manufactured by QIAGEN, and “TaqMan (trademark) MicroRNA Assay”.

manufactured by Thermo Fisher Scientific Inc. After the simultaneous detection of the plurality of microRNAs, the resulting measured values can be summed to obtain the amount of short-chain RNA.

A DNA sequencer is a method of determining the sequence of a DNA fragment base by base. In the present invention, it may be a classical method such as the Sanger method, or may be a next-generation sequencer.

In cases where “Miseq/Hiseq/NexSeq” (Illumina, Inc.), “Ion Proton/Ion PGM/lon S5/S5 XL” (Thermo Fisher Scientific Inc.), “PacBio RS II/Sequel” (Pacific Bioscience), “Nanopore Sequencer”, or the like is used as the next-generation sequencer. “MinION” (Oxford Nanopore Technologies) or the like may be used together with a commercially available measurement kit specifically designed for measurement of microRNAs.

Next-generation sequencing is a technique for determining a base sequence using a next-generation sequencer, and characterized in that a much larger number of sequencing reactions can be simultaneously carried out compared to the Sanger method (see, for example, Rick Kamps et al., Int. J. Mol. Sci., 2017,18(2), p. 308 and Int. Neurourol. J. 2016, 20 (Suppl. 2), S76-83). An example of next-generation sequencing of short-chain RNA includes the steps of adding adapter sequences having predetermined base sequences to both ends of short-chain RNA derived from a sample, or cDNA thereof, and performing reverse transcription of total RNA derived from the sample into cDNA, before or after the adapter sequence addition. After the reverse transcription, but before the sequencing step. cDNA derived from a particular target short-chain RNA may be amplified by a nucleic acid amplification method such as PCR, or concentrated by using a probe or the like. Details of the subsequent sequencing step varies depending on the type of the next-generation sequencer. Typically, the cDNA is bound to a substrate through an adapter, and sequencing reaction is carried out using the adapter sequence as a priming site for a sequencing primer, to determine and analyze the base sequence. For details of the sequencing reaction and the like, see, for example, Rick Kamps et al. (mentioned above). Finally, data output is performed. As a result, a set of sequence information (reads) obtained by the sequencing reaction, and its analysis data can be obtained. For example, in next-generation sequencing, a target microRNA can be identified based on the sequence information obtained, and the expression level of the target microRNA can be determined based on the number of reads containing the: base sequence of the target microRNA.

Unlike the measurement method described above, the method using a spectrophotometer cannot identify the length of RNA. Therefore, in this method, an RNA sample is preliminarily fractionated using an ultrafiltration column or the like to obtain short-chain RNA, followed by measurement of the short-chain RNA. Although the measurement can be carried out based on either the absorbance or a fluorescence, a measurement method using a fluorescent dye is preferred from the viewpoint of the sensitivity. For example, short-chain RNA can be fluorescently labeled using the “Quant-iT RiboGreen RNA Kit”, manufactured by Thermo Fisher Scientific Inc., and measured using “NanoDrop (registered trademark)”, manufactured by the same manufacturer.

The amount of short-chain RNA can be obtained by the above measurement methods, and, in the present invention, the “total amount” of short-chain RNA is used. The “total amount” is not necessarily limited to the amount of a particular RNA sequence, and it means the amount of a group of a plurality of kinds of short-chain RNA sequences.

By dividing the total amount of short-chain RNA contained in all extracellular vesicles obtained by the above measurement by the number of extracellular vesicles obtained in Step (b), the total amount of short-chain RNA per extracellular vesicle can be obtained. In this process, part of the extracellular vesicle fraction obtained in Step (a) may be taken, and the amount of short-chain RNA contained in the part may be measured to obtain a value instead of the “total amount of short-chain RNA contained in all extracellular vesicles”. Based on the obtained amount of short-chain RNA, the “total amount of short-chain RNA contained in all extracellular vesicles” may be calculated.

### 4. Step (d)

Step (d) in the present invention is a step of judging the subject as being affected by the neurodegenerative disease in a case where the total amount of short-chain RNA per extracellular vesicle obtained in Step (c) is larger than a total amount of short-chain RNA per extracellular vesicle similarly obtained from a body fluid sample of a healthy individual that is a control. The healthy individual herein means a subject not affected by the neurodegenerative disease. As the control, the total amount of short-chain RNA per extracellular vesicle obtained from the body fluid sample of the subject not affected by the neurodegenerative disease is used.

For example, a body fluid sample of a subject who has been shown not to be affected by the neurodegenerative disease is used as a control. An extracellular vesicle fraction is obtained therefrom in the same manner as in Step (a), and this fraction is subjected to counting of the number of extracellular vesicles simultaneously with the body fluid sample of the subject that is the detection target (Step (b)). The total amount of short-chain RNA contained in all counted extracellular vesicles is measured to obtain the total amount of short-chain RNA per extracellular vesicle (Step (c)). By using the obtained amount as a control, the subject can be judged as being affected by the neurodegenerative disease in a case where the subject shows a larger total amount of short-chain RNA per extracellular vesicle. Further, the total amount of short-chain RNA per extracellular vesicle in the control may be taken as 1, and the ratio of the total amount of short-chain RNA per extracellular vesicle in the subject to this value may be determined. In cases where the ratio is more than 1, the subject can be judged as being affected by the neurodegenerative disease.

Further, by using body fluid samples from a plurality of subjects not affected by the neurodegenerative disease as controls, the standard error of control measurement values (total amounts of short-chain RNA) can be calculated. In the comparison with the measured value of the subject, a threshold may be set, for example, at a reliability criterion of 70%, 80%, 90%, or 100%.

Further, control measurement may be carried out in advance, and the resulting measured value may be repeatedly used as a threshold. The threshold may be set as appropriate for each detection system, as a value capable of distinguishing between a control and a subject based on the total amount of short-chain RNA per extracellular vesicle in the body fluid sample. For example, in cases where the total amount of short-chain RNA is measured using “3D-Gene” (registered trademark), and the number of extracellular vesicles is counted by the nanotracking method, when a control from a single healthy individual is used, the threshold may be set, for example, at 1.1 times, 1.5 times, preferably 2 times, more preferably 2.5 times, or 5 times the total amount of short-chain RNA per extracellular vesicle in the control. When controls from a plurality of healthy individuals are used, the threshold may be set, for example, at 1.1 times, 1.5 times, preferably 2 times, more preferably 2.5 times, or 5 times the average of the total amounts of short-chain RNA per extracellular vesicle. Alternatively, the threshold may be set at the maximum value among the total amounts of short-chain RNA per extracellular vesicle in the controls from the plurality of healthy individuals, or at 1.1 times, 1.5 times, preferably 2 times, more preferably 2.5 times, or 5 times the maximum value. Since the control average value is a value for healthy individuals, the measured value can be continuously used after once it is measured. This average value may be used to set a threshold in advance. Such a threshold may be, for example, described in instructions (manufacturer's instructions) for a kit for carrying out the method of the present invention.

In cases where the total amount of short-chain RNA per extracellular vesicle obtained from a body fluid of a subject is extremely larger than the average, there is a possibility that the measurement of the total amount was incorrect. In such cases, a subject not affected by the neurodegenerative disease may be erroneously judged as being affected. In order to prevent such a problem, an upper limit value may be set, and the subject may be judged as being affected by the neurodegenerative disease in cases where the total amount of short-chain RNA is less than the upper limit value. For example, in cases where the total amount of short-chain RNA is measured using “3D-Gene” (registered trademark), and the number of extracellular vesicles is counted by the nanotracking method, when a control from a single healthy individual is used, the upper limit value may be set, for example, at 500 times, 300 times, preferably 200 times, more preferably 100 times the total amount of short-chain RNA per extracellular vesicle in the control. When controls from a plurality of healthy individuals are used, the upper limit value may be set at 500 times, 300 times, preferably 200 times, more preferably 100 times the average of the total amount of short-chain RNA per extracellular vesicle. Alternatively, the upper limit value may be set at 500 times, 300 times, preferably 200 times, more preferably 100 times the maximum value among the total amounts of short-chain RNA per extracellular vesicle in the controls from the plurality of healthy individuals.

In cases where the total amount of short-chain RNA per extracellular vesicle obtained from the body fluid of the subject is at a ratio within a particular range relative to the total amount of short-chain RNA when a single control is used, or relative to the average or maximum value when a plurality of controls are used, the subject can be judged as being affected by the neurodegenerative disease. The range may be not less than 1.1 times and less than 500 times, preferably not less than 1.5 times and less than 300 times, most preferably not less than 1.5 times and less than 100 times.

The method of judging whether or not a subject is affected by a neurodegenerative disease according to the present invention may be used in combination with imaging tests such as CT, MRI, PET, SPECT, MIBG myocardial scintigraphy, and dopamine transporter scintigraphy; tests of protein biomarkers such as amyloid β, tau, phosphorylated tau, and synuclein in spinal fluid and blood; genetic testing by PCR; and neuropsychological tests such as the MMSE test, the ECAS test, and the ALS-CBS test.

### EXAMPLES

The present invention is described more concretely by way of the following Examples. However, the scope of the present invention it not limited by the Examples.

### Example 1

### <Preparation of Extracellular Vesicle Fraction> (Step (a))

As a fraction corresponding to an extracellular vesicle fraction (including exosomes and the like) derived from a subject affected by a neurodegenerative disease, the following fraction was prepared and used.

As nerve cells, the SH-SYSY cell line (ATCC) was used. The cells were seeded in Dulbecco's modified Eagle medium (DMEM) (Nacalai Tesque, Inc.)/Ham F-12 (Ilam's F-12) medium (Nacalai Tesque, Inc.) supplemented with 10% FBS (Gibco), and retinoic acid (Fujifilm Wako Pure Chemical Corporation) was added to the medium to a final concentration of 10 μM, followed by performing culture at 37° C. and 5% CO₂ under dark conditions for 5 days. Thereafter, the cells were plated on a 6-well plate at 1×10⁵ cells per well, and the culture supernatant was removed 24 hours later, followed by washing the wells twice with 1 mL of phosphate-buffered saline (PBS) (Nacalai Tesque, Inc.). Thereafter, 3 mL of DMEM (Nacalai Tesque. Inc.)/Ham's F-12 medium (Nacalai Tesque, Inc.) was added to achieve a final amyloid β oligomer concentration of 10 μM, and then culture was performed at 37° C. for 48 hours. This treatment caused the nerve cells to suffer from neurodegeneration and to exhibit an Alzheimer's disease-like morphology. The amyloid β oligomers were prepared by dissolving amyloid β peptide (1-42) (Abcam) in DMSO) (Fujifilm Wako Pure Chemical Corporation) at 25 mM, adding hexafluoropropanol (Nacalai Tesque, Inc.) to the resulting solution to a final concentration of 1 mM, concentrating the resulting mixture to dryness using a concentration centrifuge, redissolving the resulting solid using DMSO (Fujifilm Wako Pure Chemical Corporation) to a concentration of 5 mM, adding Ham's F-12 medium (Nacalai Tesque, Inc.) thereto to adjust the final concentration to 100 μM, and then incubating the resulting mixture at 4° C. for 24 hours before the use. After the culture, the cell supernatant was collected, and then centrifuged at 10,000×G at 4° C. for 30 minutes, followed by collecting the supernatant.

As an affinity method, the “MagCapture (trademark) Exosome Isolation Kit PS” (Fujifilm Wako Pure Chemical Corporation) was used according to the recommended protocol, to obtain an extracellular vesicle fraction (including exosomes and the like) from 1 ml, of the cell culture supernatant after the above-described centrifugation.

On the other hand, as a control corresponding to an extracellular vesicle fraction derived from a subject not affected by the neurodegenerative disease, an extracellular vesicle fraction was prepared in the same manner as described above except that the amyloid β oligomers were not added.

### <Counting of Extracellular Vesicles> (Step (b))

The number of extracellular vesicles contained in the extracellular vesicle fraction prepared in the Step (a) was counted by the nanotracking method. The number of extracellular vesicles was counted for a ⅕ portion of the extracellular vesicle fraction obtained in Step (a), using a nanoparticle tracking analyzer “NanoSight NS 300” (NanoSight Ltd.), and analysis was carried out using NTA 3.2 (NanoSight Ltd.). The results are shown in the upper panel of FIG. 1. Data were obtained from n=3. The ordinate of the graph shows the number of exosome particles (with a diameter of 80 to 120 nm) (/mL). The bar graph shows the mean ±standard deviation.

The average number of exosomes prepared with the addition of the amyloid β oligomers was 6.68×10⁶ /mL ((+) in the right side of the upper panel of FIG. 1). On the other hand, the average number of exosomes prepared without the addition of the amyloid β oligomers, provided as a control, was 2.61×10⁶ /mL ((−) in the left side of the upper panel of FIG. 1).

### <Measurement of Total Amount of Short-Chain RNA in Extracellular Vesicles> (Step (c))

The total amount of short-chain RNA contained in all extracellular vesicles contained in the extracellular vesicle fraction prepared in the Step (a) was obtained as follows. From a ⅘ portion of the extracellular vesicle fraction obtained in Step (a), total RNA was obtained using the RNA extraction reagent contained in the “3D-Gene (registered trademark) RNA extraction reagent from liquid sample kit” (Toray Industries, Inc.) according to the protocol recommended by the manufacturer. The total RNA obtained was subjected to fluorescent labeling of microRNA using the “3D-Gene (registered trademark) miRNA Labeling kit” (Toray Industries, Inc.) according to the protocol recommended by the manufacturer. Hybridization and subsequent washing were carried out under high stringent conditions using, as an oligo DNA chip, a “3D-Gene (registered trademark) Human miRNA Oligo chip” (Toray Industries, Inc.), which contains probes having sequences complementary to 2632 kinds of microRNAs out of the microRNAs deposited in miRBase release 22 (http://www.mirbase.org/), according to the protocol recommended by the manufacturer. The DNA chip was scanned using a “3D-Gene (registered trademark) Scanner” (Toray Industries, Inc.) to obtain an image, and then digitalization of the fluorescence intensity was performed using “3D-Gene (registered trademark) Extraction (Toray Industries, Inc.)” to obtain the gene expression levels of the comprehensive microRNAs. The sum of the microRNA gene expression levels (linear values) detected was obtained as the total amount of microRNA.

The total amount of microRNA contained in the exosomes prepared with the addition of the amyloid β oligomers was 2.46×10⁵. The total amount of microRNA contained in the exosomes prepared without the addition of the amyloid β oligomers, provided as the control, was 3.10×10⁵.

The total amount of microRNA obtained was divided by the number of exosomes obtained in the Step (b), to calculate the amount of microRNA per exosome. The total amount of microRNA per exosome in the exosomes prepared with the addition of the amyloid β oligomers was 0.037. On the other hand, the total amount of microRNA per exosome in the exosomes prepared without the addition of the amyloid β oligomers, provided as the control, was 0.119.

### <Judgment of Presence or Absence of Neurodegeneration> (Step (d))

Based on the total amount of microRNA per exosome obtained in the Step (c), the relative ratio of the total amount of microRNA in the case with the addition of the amyloid β oligomers was determined compared to the total amount of microRNA in the case without addition of the amyloid β oligomers, which was taken as 1. The results are shown in the lower panel of FIG. 1. Data were obtained from n=3. The ordinate of the graph shows the relative ratio of the total amount of microRNA per exosome.

As a result, the total amount of microRNA per exosome significantly increased in the case with the addition of amyloid β ((+) in the right side of the lower panel of FIG. 1) compared to the case without the addition of the amyloid β oligomers ((−) in the left side of the lower panel of FIG. 1) (P value<0.005).

These results indicate that comparison of the total amount of microRNA per exosome with those of controls enables judgment of whether or not a subject is, affected by neurodegeneration.

### Example 2

### <Preparation of Extracellular Vesicle Fraction> (Step (a))

As a fraction corresponding to an extracellular vesicle fraction (including exosomes and the like) derived from a subject affected by a neurodegenerative disease, the following fraction was prepared and used.

Sera obtained from three cases of healthy individuals. Alzheimer's disease patients, or ALS patients with informed consent, purchased from BizComJapan, Inc. were used (Table 1).

TABLE 1

| Diagnosis | Age | Gender | MMSE |
|---|---|---|---|
| CONTROL | 66 | M | 29 |
| CONTROL | 82 | F | 29 |
| CONTROL | 58 | M | 29 |
| AD | 69 | F | 19 |
| AD | 68 | M | 23 |
| AD | 68 | M | 17 |
| ALS | 61 | M | 30 |
| ALS | 57 | F | 30 |
| ALS | 73 | M | 30 |

CONTROL: Healthy individual;
AD: Alzheimer's disease;
ALS: Amyotrophic lateral sclerosis;
MMSE: Mini-Mental state examination

An immunoprecipitation method was carried out using a biotin-labeled anti-L1CAM antibody (Invitrogen) and the ExoCap (trademark) Streptavidin Kit (MBL) according to the protocol recommended by the ExoCap (trademark) Streptavidin Kit, wherein 1 mL each of the above sera was centrifuged at 100,000×G for 10 minutes at 4° C. to obtain a supernatant, from which an extracellular vesicle fraction (including exosomes and the like) was obtained.

### <Counting of Extracellular Vesicles> (Step (b))

The number of extracellular vesicles contained in the extracellular vesicle fraction prepared in the Step (a) was detected by an antigen-antibody reaction method. To a ⅕ portion of the extracellular vesicle fraction obtained in Step (a), a solution prepared by 500-fold dilution of an HIRP-labeled anti-CD63 antibody (Santa Cruz Biotechnology) was added, and the resulting mixture was stirred at 4° C. for 4 hours. Thereafter, the extracellular vesicle value (luminescence intensity) was detected by measurement using a microplate reader (MOLECULAR DEVICES) according to the protocol recommended by the ExoCap (trademark) Streptavidin Kit. The extracellular vesicle value in the case of the preparation from the sera of the Alzheimer's disease patients was 7.51×10⁵. The extracellular vesicle value in the case of the preparation from the sera of the ALS patients was 7.11×10⁵. On the other hand, the extracellular vesicle value in the case of the preparation from the sera of the healthy individuals was 9.42×10⁵. The results in terms of the relative values are shown in the upper panel of FIG. 2. Data were obtained from n=3. The ordinate of the graph shows the relative value of the number of exosomes to the average in the healthy individuals. The bar graph shows the relative value calculated taking the average in the healthy individuals as 1. The average number of the exosomes prepared from the Alzheimer's disease patients (relative value to that in the healthy individuals) was 0.798 (the middle (AD) of the upper panel of FIG. 2). The average number of the exosomes prepared from the ALS patients (relative value to the average number in the healthy individuals) was 0.755 (the right side (ALS) of the upper panel of FIG. 2).

### <Measurement of Total Amount of Short-Chain RNA in Extracellular Vesicles> (Step (c);

The total amount of short-chain RNA contained in all extracellular vesicles contained in the extracellular vesicle fraction prepared in the Step (a) was obtained as follows. From a ⅘ portion of the extracellular vesicle fraction obtained in Step (a), total RNA was obtained using the RNA extraction reagent contained in the “trademark) RNA extraction reagent from liquid sample kit” (Toray Industries, Inc.) according to the protocol recommended by the manufacturer. The total RNA obtained was subjected to fluorescent labeling of microRNA using the “3D-Gene (registered trademark) miRNA Labeling kit” (Toray Industries, Inc.) according to the protocol recommended by the manufacturer. Hybridization and subsequent washing were carried out under high stringent conditions using, as an oligo DNA chip, a “3D-Gene (registered trademark) Human miRNA Oligo chip” (Toray Industries, Inc.), which contains probes having sequences complementary to 2632 kinds of microRN. As out of the microRNAs deposited in miRBase release 22 (http://www.mirbase.org/), according to the protocol recommended by the manufacturer. The DVA chip was scanned using a “3D-Gene (registered trademark) Scanner” (Toray Industries, Inc.) to obtain an image, and then digitalization of the fluorescence intensity was performed using “3D-Gene (registered trademark) Extraction (Toray Industries, Inc.)” to obtain the gene expression levels of the comprehensive microRNAs. The sum of the microRNA gene expression levels (linear values) detected was obtained as the total amount of microRNA.

The total amount of microRNA contained in the exosomes prepared from the sera of the Alzheimer's disease patients was 9.56×10³. The total amount of microRNA contained in the exosomes prepared from the sera of the ALS patients was 7.74×10³. The total amount of microRNA contained in the exosomes prepared from the sera of the healthy individuals was 8.13×10³.

The total amount of microRNA obtained was divided by the value of the antigen-antibody reaction method obtained in the Step (b), to calculate the amount of microRNA per exosome. The total amount of microRNA per exosome in the serum exosomes of the healthy individuals was 0.011. On the other hand, the total amount of microRNA per exosome in the serum exosomes of the Alzheimer's disease patients was 0.019. The total amount of microRNA per exosome in the serum exosomes of the ALS patients was 0.075.

### <Judgment of Presence or Absence of Neurodegeneration> (Step (d))

Based on the total amount of microRNA per exosome obtained in the Step (c), the relative ratios of the total amount of microRNA in the Alzheimer's disease patients and the ALS patients were determined compared to that of the healthy individuals, which was taken as 1. The results are shown in the lower panel of FIG. 2. Data were obtained from n=3. The ordinate of the graph shows the total amount of microRNA per exosome (relative value) as compared to the average in the healthy individuals. The bar graph shows the relative value calculated taking the average in the healthy individuals as 1. The total amount of microRNA per exosome prepared from the Alzheimer's disease patients (relative value to that in the healthy individuals) was 1.705 (the middle (AD) of the lower panel of FIG. 2). The total amount of microRNA per exosome prepared from the ALS patients (relative value to that in the healthy individuals) was 6.641 (the right side (ALS) of the lower panel of FIG. 2). The total amount of microRNA per exosome was not less than 1.5 times larger in the Alzheimer's disease patients, and not less than 5 times larger in the ALS patients.

These results indicate that comparison of the total amount of microRNA per exosome with those of control healthy individuals enables judgment of whether or not a subject is affected by neurodegeneration.

1. A method of detecting whether or not a subject is affected by a neurodegenerative disease, the method comprising the steps of:
(a) preparing an extracellular vesicle fraction from a body fluid sample of the subject;
(b) counting the number of extracellular vesicles contained in the extracellular vesicle fraction obtained in Step (a), to obtain the number of the extracellular vesicles;
(c) measuring the total amount of short-chain RNA contained in all extracellular vesicles counted in Step (b), to obtain the total amount of short-chain RNA per extracellular vesicle; and
(d) judging the subject as being affected by the neurodegenerative disease in a case where the total amount of short-chain RNA per extracellular vesicle obtained in Step (c) is larger than a total amount of short-chain RNA per extracellular vesicle obtained from a body fluid sample of a healthy individual.

2. The method according to claim 1, wherein in the Step (d), an average of total amounts of short-chain RNA per extracellular vesicle preliminarily obtained from body fluid samples of a plurality of healthy individuals is calculated, and the subject is judged as being affected by the neurodegenerative disease in a case where the total amount of short-chain RNA per extracellular vesicle obtained from the body fluid sample of the subject is not less than 1.5 times, and less than 100 times the average.

3. The method according to claim 1, wherein in the Step (a), the prepared extracellular vesicle fraction contains an extracellular vesicle having a diameter of 30 nm to 200 nm.

4. The method according to claim 1, wherein in the Step (a), CD9, CD63, CD81, Tim4, or L1CAM protein is present on the surface of a prepared extracellular vesicle(s).

5. The method according to claim 1, wherein in the Step (b), the short-chain RNA has a length of 15 bases or more and 200 bases or less.

6. The method according to claim 1, wherein the short-chain RNA is a microRNA.

7. The method according to claim 1, wherein the body fluid sample is blood, serum, plasma, or cerebrospinal fluid.

8. The method according to claim 1, wherein in the Step (a), the extracellular vesicle fraction is prepared by a method selected from the group consisting of a centrifugation method, an immunoprecipitation method, a polymer precipitation method, a lipid affinity method, liquid chromatography, size exclusion chromatography, an ultrafiltration method, and combinations thereof.

9. The method according to claim 1, wherein in the Step (b), the number of the extracellular vesicles is counted by a tracking method, an antigen-antibody reaction method, or a flow cytometry method.

10. The method according to claim 1, wherein in the Step (c), the total amount of short-chain RNA contained in the extracellular vesicles is measured using a spectrophotometer, electrophoresis, microarray, PCR, or a DNA sequencer.

11. The method according to claim 1, wherein the neurodegenerative disease is Alzheimer's disease, dementia with Lewy Bodies, frontotemporal dementia, amyotrophic lateral sclerosis (ALS), or Parkinson's disease.

12. A kit for detecting whether or not a subject is affected by a neurodegenerative disease by the method according to claim 1, the kit comprising:
means for preparing an extracellular vesicle fraction from a body fluid sample of the subject;
means for counting the number of extracellular vesicles contained in the prepared extracellular vesicle fraction, to obtain the number of the extracellular vesicles; and
means for measuring the total amount of short-chain RNA contained in all counted extracellular vesicles, to obtain the total amount of short-chain RNA per extracellular vesicle.

13. The kit according to claim 12, wherein
the means for preparing the extracellular vesicle fraction from the body fluid sample of the subject comprises an immobilized antibody or antigen-binding fragment thereof, prepared by immobilization of an antibody that specifically binds to a target surface antigen on extracellular vesicles or immobilization of an antigen-binding fragment of the antibody;
the means for counting the number of extracellular vesicles contained in the prepared extracellular vesicle fraction, to obtain the number of the extracellular vesicles, comprises a labeled antibody or antigen-binding fragment of the antibody, which antibody specifically binds to a target surface antigen on extracellular vesicles; and
the means for measuring the total amount of short-chain RNA contained in all counted extracellular vesicles, to obtain the total amount of short-chain RNA per extracellular vesicle, comprises a chip containing nucleic acid probes that hybridize with a plurality of known microRNAs.

14. A system for detecting whether or not a subject is affected by a neurodegenerative disease by the method according to claim 1, the system comprising:
means for preparing an extracellular vesicle fraction from a body fluid sample of the subject;
means for counting the number of extracellular vesicles contained in the prepared extracellular vesicle fraction, to obtain the number of the extracellular vesicles; and
means for measuring the total amount of short-chain RNA contained in all counted extracellular vesicles, to obtain the total amount of short-chain RNA per extracellular vesicle.
